(19) United States
(12) Patent Application Publication
(10) Pub. No.: US 2023/0150864 A1
(43) Pub. Date: May 18, 2023

(54) GLASS COMPOSITION

(71) Applicant: IR SCIENTIFIC INC., Halifax (CA)

(72) Inventors: Daniel BOYD, Upper Tantallon (CA); Kathleen Naomi MACDONALD-PARSONS, Bedford (CA); Geralda Effie BOUDREAU, Halifax (CA)

(21) Appl. No.: 17/910,296

(22) PCT Filed: Mar. 8, 2021
(86) PCT No.: PCT/CA2021/050309

§ 371 Date: Sep. 8, 2022

Related U.S. Application Data
(60) Provisional application No. 62/987,192, filed on Mar. 9, 2020.

(51) Int. Cl. C03C 3/14; C03C 3/247; C03C 4/00; A61K 8/24; A61Q 11/00

(52) U.S. Cl. CPC C03C 3/14; C03C 3/247; C03C 4/0021; A61K 8/24; A61Q 11/00

(57) ABSTRACT

The present disclosure provides glass compositions that include from about 20 mol % to 45 mol % of B₂O₃; and from about 10 mol % to about 80 mol % of one or more glass components selected from the group consisting of CaO and MgO. The glass compositions also include less than 0.1 mol % CdO. The glass compositions may include one or more of Na₂O, K₂O, and a phosphate source, where the B2O3 and the phosphate source total about 60 mol % or less. The glass compositions may include a source of fluoride. The glass composition may be used to desensitize dentin. The present disclosure also provides dentin-desensitizing compositions, as well as methods and uses of the disclosed glass compositions.

### CROSS REFERENCE TO RELATED APPLICATIONS

This patent application claims the benefit of priority from U.S. Application No. 62/987,192 filed Mar. 9, 2020, the contents of which are incorporated herein by reference in their entirety.

### FIELD

The present disclosure relates to glass compositions that may be formulated for dentin-desensitizing compositions.

### BACKGROUND

The following paragraphs are not an admission that anything discussed in them is prior art or part of the knowledge of persons skilled in the art.

Dentin sensitivity is dental pain that arises from exposed dentin surfaces in response to stimuli, such as thermal, evaporative, tactile, osmotic, chemical or electrical. Dentin sensitivity may be caused by gingival recession (receding gums) with exposure of root surfaces, loss of the cementum layer and smear layer, tooth wear, acid erosion, periodontal root planing, or dental bleaching.

Dentine contains many thousands of microscopic tubular structures that radiate outwards from the pulp. Changes in the flow of the plasma-like biological fluid present in the dentinal tubules can trigger mechanoreceptors present on nerves located at the pulpal aspect, thereby eliciting a pain response. This hydrodynamic flow can be increased by cold, air pressure, drying, sugar, sour (dehydrating chemicals), or forces acting onto the tooth. Hot or cold food or drinks, and physical pressure are typical triggers in those individuals with teeth sensitivity.

There is no universally accepted, gold-standard treatment which reliably relieves the pain of dental hypersensitivity in the long term. However, treatments can be divided into in-office (e.g. intended to be applied by a dentist or dental therapist), or treatments which can be carried out at home, available over-the-counter or by prescription.

The purported mechanism of action of these treatments is either occlusion of dentin tubules, or desensitization of nerve fibres/blocking the neural transmission.

### INTRODUCTION

The following introduction is intended to introduce the reader to this specification but not to define any invention. One or more inventions may reside in a combination or sub-combination of the apparatus elements or method steps described below or in other parts of this document. The inventors do not waive or disclaim their rights to any invention or inventions disclosed in this specification merely by not describing such other invention or inventions in the claims.

E. I. Kamitsos in J. Phys. Chem. 1989, 93, 1604-1611 discloses alkali-metal borate glasses of the formula xM₂O.(1−x)B₂O₃, where M is Li, Na, K, Rb, Cs and where x is from 0 to 0.4. That is, Kamitsos teaches alkali-metal borate glasses with at least 60 mol % B₂O₃.

Y. D. Yiannopolous and E. I Kamitsos in Phys. Chem. Glasses, 2001, 42(3), 164-72 studied alkaline earth borate glasses of the formula xMO.(1−x)B₂O₃, where M is Mg, Ca, Sr, Ba and where x is from 0.15 to 0.55. In Table 1, Yiannopolous and Kamitsos state that the glass forming region when M is Mg is when x is from 0.45 to 0.55, and the glass forming region when M is Ca is when x is from 0.33 to 0.50.

One or more described embodiments attempt to address or ameliorate one or more shortcomings involved with dentin-desensitizing compositions that include particulate material that occludes dentin tubules. In some embodiments, the disclosed particulate material substantially degrades over a period between 12 and 24 hours under environmental conditions. In some embodiments, the disclosed particulate material provides a controlled release of fluoride over the same time period. In some embodiments, the disclosed particulate material provides a controlled release of potassium over the same time period.

Glass compositions according to the present disclosure include from about 20 mol % to 45 mol % of B₂O₃; and from about 10 mol % to about 80 mol % of one or more glass components selected from the group consisting of CaO and MgO. Glass compositions according to the present disclosure also include less than 0.1 mol % CdO. The glass compositions may additionally include less than 0.1 mol % of CuO; less than 0.1 mol % of Li₂O; less than 0.1 mol % of Rb₂O; less than 0.1 mol % of BaO; less than 0.1 mol % of SrO; less than 0.1 mol % of SiO₂; or any combination thereof.

Glass compositions according to the present disclosure may include one or more glass components selected from the group consisting of Na₂O, K₂O, and a phosphate source. When the composition includes a phosphate source, the total moles of B₂O₃ and the phosphate source is less than or equal to about 60 mol %. The phosphate source may be P₂O₅, NaH₂PO₄, Na₂HPO₄, Na₃PO₄, KH₂PO₄, K₂HPO₄, K₃PO₄, or any combination thereof.

Glass compositions according to the present disclosure may, additionally or alternatively, include up to about 45 mol % of CaF₂, SnF₂, NaF, KF, Na₂PO₃F, or a combination thereof.

One exemplary composition according to the present disclosure includes about 43 mol % of B₂O₃, about 21 mol % MgO, about 21 mol % CaO, and about 15 mol % Na₂O; such as including 43.0 mol % of B₂O₃, 20.7 mol % MgO, 20.7 mol % CaO, and 15.6 mol % Na₂O.

Glass compositions according to the present disclosure may be in the form of a bulk glass, or a particulate material prepared from a bulk glass. The chemical formulations are the same between a bulk glass and the particulate material formed therefrom. The particulate material may include particles that are from about 1 to about 50 μm in size. At least 75% of the particles may be smaller than 50 μm in size, at least 5% of the particles may be smaller than 7 μm in size, or both.

Some exemplary glass compositions formulated as particulate material may lose at least 5 mass % within 24 hours when exposed to a buffered saline solution. Some exemplary compositions may lose at least 20, at least 40, at least 60, or at least 80 mass % within 24 hours when exposed to a buffered saline solution. Other exemplary glass compositions formulated as particulate material may lose less than 5 mass % after being exposed to a buffered saline solution for 24 hours.

Glass compositions according to the present disclosure may be formulated into a dentin-desensitizing composition, such as a toothpaste, a prophylaxis paste, a tooth varnish, a mouthwash, a dental gel, or a bonding agent. Dentin-desensitizing compositions according to the present disclosure are substantially water-free.

Glass compositions according to the present disclosure may be used for desensitizing dentin, such as in methods that include applying to an individual's dentin: a toothpaste, a prophylaxis paste, a tooth varnish, a mouthwash, a dental gel, or a bonding agent according to the present disclosure.

### DETAILED DESCRIPTION

Glass compositions according to the present disclosure include from about 20 mol % to 45 mol % of B₂O₃; and from about 10 mol % to about 80 mol % of one or more glass components selected from the group consisting of CaO and MgO. Glass compositions according to the present disclosure also include less than 0.1 mol % CdO.

Glass compositions according to the present disclosure may include one or more glass components selected from the group consisting of Na₂O, K₂O, and a phosphate source. When the composition includes a phosphate source, the total moles of B₂O₃ and the phosphate source is less than or equal to about 60 mol %. For example, exemplary compositions may include any combination of B₂O₃ and phosphate source where the molar amounts total about 25 mol % to about 30 mol %, about 30 mol % to about 35 mol %, about 35 mol % to about 40 mol %, about 40 mol % to about 45 mol %, about 45 mol % to about 50 mol %, about 50 mol % to about 55 mol %, or about 55 mol % to about 60 mol %. Additionally, the phosphate source may be, for example, less than 40 mol %, less than 35 mol %, less than 30 mol %, less than 25 mol %, less than 20 mol %, less than 15 mol %, less than 10 mol %, or less than 5 mol %. The phosphate source may be P₂O₅, NaH₂PO₄, Na₂HPO₄, Na₃PO₄, KH₂PO₄, K₂HPO₄, K₃PO₄, or any combination thereof.

Glass compositions according to the present disclosure may include up to about 45 mol % of CaF₂, SnF₂, NaF, KF, Na₂PO₃F, or a combination thereof.

The glass composition may be formulated as a particulate material that includes particles that are from about 1 to about 50 μm in size. The glass composition may include at least some particles that are sized to luminally occlude dentinal tubules, thereby desensitizing the dentin. In the context of the present disclosure, a particle sized to luminally occlude a dentinal tubule should be understood to mean that the particle sits in or on top of the dentinal tubule, reducing the movement of the dentinal fluid. The glass composition may include at least some particles that are sized to provide surface occlusion of dentinal tubules, thereby desensitizing the dentin.

It should be understood that the expression “about X mol % to about Y mol % of one or more glass components” refers to the total mol % of the glass components, and does not refer to the mol % percent of each individual component. For example, a glass composition according to the present disclosure could include 5 mol % of each of CaO and MgO in order to provide 10 mol % of the one or more glass components selected from the group consisting of CaO and MgO.

It should be understood that any disclosure of a contemplated range of values is also a disclosure of any value or subrange within the recited range, including endpoints. For example, a contemplated rate of “1 to 100” is also a disclosure of, for example: 1, 10, 25 to 57, 32 to 84, 25 to 84, and 32 to 75.

It should be understood that “about X mol %” refers to any value that is within ±2% of the reported percentage. For example, “about 10 mol %” would refer to values from 8 mol % to 12 mol % since all those values would be within ±2% of the reported 10%; and “about 50 mol %” would refer to values from 48 mol % to 52 mol % since all those values would be within ±2% of the reported 50%.

It should be understood that any contemplated range of values is also a disclosure of any value or subrange within the recited range, including endpoints. For example, a contemplated rate of “1 to 100” is also a disclosure of, for example: 1, 10, 25 to 57, 32 to 84, 25 to 84, and 32 to 75.

It should be understood that “about X μm” in the context of particle size is determined based on accepted tolerances as per ASTM E-11 for a test sieve of the noted size. For example, the accepted tolerance for a 50 μm test sieve is 3 μm. Accordingly, “about 50 μm” refers to particles that are from 47 μm to 53 μm in size. In another example, the accepted tolerance for a 35 μm test sieve is 2.6 μm. Accordingly, “about 35 μm” refers to particles that are from 32.4 μm to 38.6 μm in size. The ASTM accepted tolerance for a 25 μm sieve is 2.2 μm. For test sieves without a standard, accepted tolerance (such as test sieves below 20 μm), the expression “about X μm” refers to ±15% for sizes from 5 to 15 μm, and ±50% for sizes less than 5 μm. For example “about 1 μm” refers to particles that are from 0.5 to 1.5 μm in size.

It should be understood that a “glass” according to the present disclosure is a ceramic material that displays a glass transition temperature above room temperature, and whose principal phase is primarily amorphous, such as at least 50% amorphous, at least 75% amorphous, at least 90% amorphous, at least 95% amorphous, or at least 97% amorphous. In some examples, a glass according to the present disclosure is substantially free or completely free, of identifiable crystalline species.

In the context of the present disclosure, an “optional” component of the glass composition is a component that may be present in some exemplary compositions and absent in other exemplary compositions. Reference to more than one “optional” component should be understood to mean that a composition according to the present disclosure may include none, one, or any combination of the optional components. For example, glass compositions according to the present disclosure (a) optionally include one or more glass components selected from the group consisting of Na₂O, K₂O, and a phosphate source; and (b) optionally include a source of fluoride. Accordingly, the present disclosure contemplates exemplary glass compositions that: (i) lack all of the optional components; (ii) include one or more glass components selected from the group consisting of Na₂O, K₂O, and a phosphate source, but lack a source of fluoride; (iii) include a source of fluoride, but lack Na₂O, K₂O, and a phosphate source; and (iv) include one or more glass components selected from the group consisting of Na₂O, K₂O, and a phosphate source, as well as a source of fluoride.

Glass compositions that include CaO, MgO, a phosphate source, or a combination thereof may help form a precipitate and/or mineralize apatites, such as hydroxyapatite which is the major component of tooth enamel. Forming a precipitate or mineralizing apatites in or around the dentinal tubules may form a protective precipitate and further decrease dentin sensitivity.

Glass compositions that include potassium (such as K₂O, KH₂PO₄, K₂HPO₄, K₃PO₄, or KF) release the potassium when the glass degrades. Without wishing to be bound by theory, it is believed that released potassium blocks or reduces the action potential generated in intradental nerves, thereby reducing dentinal sensitivity.

A glass composition according to the present disclosure may include, for example, from about 10 mol % to about 80 mol % of: (a) CaO; (b) MgO; (c) a combination of CaO and MgO; (d) a combination of (i) CaO and (ii) Na₂O and/or K₂O; (e) a combination of (i) MgO and (ii) Na₂O and/or K₂O; (f) a combination of (i) CaO, (ii) MgO and (iii) Na₂O and/or K₂O; (g) a combination of (i) CaO or MgO and (ii) a phosphate source; (h) a combination of (i) CaO, (ii) MgO and (iii) a phosphate source; (i) a combination of (i) CaO or MgO, (ii) a phosphate source, and (iii) Na₂O and/or K₂O; or (j) a combination of (i) CaO, (ii) MgO, (iii) a phosphate source, and (iv) Na₂O and/or K₂O. Any of these exemplary compositions may additionally include a source of fluoride.

In the context of the present disclosure, it should be understood that glass compositions that include Na₂O, K₂O, a phosphate source, or combinations thereof must still include at least 10 mol % of CaO, MgO, or a combination thereof, and when the composition includes a phosphate source, the total moles of B₂O₃ and the phosphate source must still be less than or equal to about 60 mol %. For example, reference to a composition that includes from “about 10 mol % to about 80 mol % of a combination of (i) CaO, (ii) MgO and (iii) a phosphate source”, should be understood to refer to any combination of CaO, MgO and a phosphate source where at least 10 mol % of the composition is a combination of CaO and MgO, the combination of CaO, MgO and the phosphate source is from 10 mol % to 80 mol %, and the combination of B₂O₃ and the phosphate source is less than or equal to about 60 mol %. Similarly, reference to a composition that includes “about 10 mol % to about 80 mol % of a combination of (i) CaO or MgO, (ii) a phosphate source, and (iii) Na₂O and/or K₂O” should be understood to be a reference to any combination of: (i) CaO or MgO, plus (ii) a phosphate source, plus (iii) Na₂O, K₂O, or a combination of Na₂O and K₂O, where at least 10 mol % of the composition is CaO or MgO, the combination of CaO or MgO, the phosphate source, Na₂O and K₂O, is from 10 mol % to 80 mol %, and the combination of B₂O₃ and the phosphate source is less than or equal to about 60 mol %.

Some exemplary glass compositions according to the present disclosure include a source of fluoride, such as up to about 45 mol % of CaF₂, SnF₂, NaF, KF, Na₂PO₃F, or a combination thereof. Including fluoride in the glass composition results in fluoride being released when the glass degrades. The released fluoride may form fluoridated apatites, such as fluorapatite (Ca₅(PO₄)₃F) in or around the dentinal tubules, which may form a protective precipitate and further decrease dentin sensitivity.

Compositions that include CaF₂ or SnF₂ provide twice the amount of fluoride per mole of starting material compared to compositions that use NaF, Na₂PO₃F, or KF. In some examples, the glass includes less than 30 mol % of CaF₂, SnF₂, or a combination thereof.

In some examples, the glass composition may include about 2 mol % to about 15 mol % of CaF₂, SnF₂, NaF, KF, Na₂PO₃F, or a combination thereof. In some examples, glass compositions according to the present disclosure may include one or more of: NaF, KF, and CaF₂, such as in an amount from about 5 mol % to about 15 mol %.

In some examples, a glass composition according to the present disclosure includes sufficient fluoride that 0.1 g of the particulate material releases the fluoride into 10 mL of a buffered saline solution at an average rate of about 0.5 ppm/hr to about 2000 ppm/hr over 1, 2, 4, 8, 12, 18 or 24 hours. In the context of the present disclosure, ppm is measured as mass/volume when determining the release rate of fluoride. In particular examples, the glass composition includes sufficient fluoride that about 4 to about 6 ppm of fluoride is released per hour over 1 hour.

Glass compositions according to the present disclosure may include Na₂O, CaO, and MgO in a molar ratio of 1.0:0.5 to 2.5:0.5 to 2.5 (Na₂O:CaO:MgO). In some examples, the glass composition includes: a) from about 16 mol % to about 22 mol % Na₂O, from about 11 mol % to about 17 mol % CaO, and from about 16 mol % to about 22 mol % MgO; b) from about 14 mol % to about 20 mol % Na₂O, from about 14 mol % to about 20 mol % CaO, and from about 16 mol % to about 22 mol % MgO; c) from about 11 mol % to about 17 mol % Na₂O, from about 16 mol % to about 22 mol % CaO, and from about 16 mol % to about 22 mol % MgO; or d) from about 13 mol % to about 19 mol % Na₂O, from about 18 mol % to about 24 mol % CaO, and from about 18 mol % to about 24 mol % MgO.

Glass compositions according to the present disclosure may include B₂O₃, MgO, CaO, Na₂O, and K₂O in a molar ratio where (B₂O₃+MgO):(CaO+Na₂O+K₂O) is greater than 1.0, such as greater than 1.15 or greater than 1.30.

In some exemplary glass compositions according to the present disclosure, the composition includes: (a) at least 54 mol %, such as at least 57 mol %, of a combination of B₂O₃ and MgO; (b) at least 33 mol %, such as at least 40 mol % or at least 50 mol %, of a combination of CaO and MgO; (c) at least 7 mol %, such as at least 15 mol % or at least 30 mol %, of a combination of N₂O and K₂O; (d) or any combination thereof. The exemplary glass compositions may include less than 0.1 mol % phosphate. The exemplary glass compositions may consist essentially of B₂O₃, one or both of Na₂O and K₂O, and one or both of CaO and MgO.

Exemplary glass compositions according to the present disclosure include B₂O₃, one or both of Na₂O and K₂O, and one or both of CaO and MgO in amounts according to any one of the compositions listed in Table 1A and 1B.

In some exemplary glass compositions according to the present disclosure, the composition includes from about 25 mol % to about 43 mol % B₂O₃; from about 14 mol % to about 21 mol % CaO; from about 19 mol % to about 29 mol % MgO; from about 9 mol % to about 15 mol % Na₂O; and from about 9 mol % to about 15 mol % of NaF, KF, CaF₂, or any combination thereof.

In one particular example of a glass composition according to the present disclosure, the composition includes about 43 mol % of B₂O₃, about 21 mol % MgO, about 21 mol % CaO, and about 15 mol % Na₂O; such as including 43.0 mol % of B₂O₃, 20.7 mol % MgO, 20.7 mol % CaO, and 15.6 mol % Na₂O.

In some exemplary glass compositions according to the present disclosure, the composition includes from about 25 mol % to about 45 mol %, such as from about 41 mol % to about 45 mol %, of B₂O₃; from about 10 mol % to about 23 mol %, such as from about 13 mol % to about 23 mol %, of CaO; from about 10 mol % to about 30 mol %, such as from about 18 mol % to about 23 mol %, of MgO; and from about 8 mol % to about 22 mol %, from about 13 mol % to about 22 mol %, of Na₂O. The compositions may optionally include from about 8 mol % to about 15 mol % of NaF, KF, CaF₂, or any combination thereof.

In some exemplary glass compositions according to the present disclosure, the composition includes from about 29 mol % to about 45 mol % of B₂O₃; from about 5 mol % to about 22 mol % of CaO; from about 1 mol % to about 22 mol % of MgO; from 0 mol % to about 15 mol % of K₂O; and from about 5 mol % to about 18 mol % of Na₂O.

Glass compositions according to the present disclosure may include less than 0.1 mol % of ZnO, such as substantially no ZnO; less than 0.1 mol % of CuO; less than 0.1 mol % of Li₂O; less than 0.1 mol % of Rb₂O; less than 0.1 mol % of BaO; less than 0.1 mol % of SrO; less than 0.1 mol % of SiO₂; or any combination thereof.

Particle Size Distribution

A glass composition according to the present disclosure may be formulated as a particulate material that includes particles that are from about 1 to about 50 μm in size. Such glass compositions may be referred to as “particulate glass compositions”. In some examples, at least some of the particles are sized to sit in or on top of a dentinal tubule. Dentinal tubules have a natural variation in diameter and are primarily from about 0.5 to about 8 μm in size, for example, from about 0.5 to about 5 μm in size. Accordingly, glass compositions of the present disclosure that are formulated as a particulate material may be used for desensitizing dentin, which may temporarily reduce pain associated with sensitive teeth.

In some examples, at least 75% of the particles making up the particulate material are smaller than 50 μm in size. In other examples, at least 85% or at least 95% of the particles are smaller than 50 μm in size. In some examples, at least 5% of the particles making up the particulate material are smaller than 7 μm in size.

In particular examples, the particulate material is made up of a plurality of particles where at least 5% of the particles are smaller than 35 μm in size, at least 5% of the particles are smaller than 15 μm in size, and at least 5% of the particles are smaller than 7 μm in size.

In particular examples, the particulate material is made up of a plurality of particles where at least 5% of the particles are from about 15 μm to about 35 μm in size, at least 5% of the particles are from about 6 μm to about 15 μm in size, and at least 5% of the particles are from about 3 μm to about 7 μm in size.

In some particular examples, the particulate material is made up of a plurality of particles where the particle size distribution is Dx10 of about 5 um, Dx50 of about 15 um, and Dx90 of about 30 um.

Degradation

Some particulate glass compositions according to the present disclosure may degrade under physiological conditions, for example particulate glass compositions according to the present disclosure may lose at least 5 mass % within 24 hours when exposed to a buffered saline solution. In some examples, the glass composition may lose at least 20 mass %, at least 40 mass %, at least 60 mass %, or at least 80 mass % within 24 hours when exposed to the buffered saline solution.

Other particulate glass compositions according to the present disclosure may resist degradation under physiological conditions, for example losing less than 5 mass % after being exposed to a buffered saline solution for 24 hours.

Surface Microhardness and Remineralization

Glass compositions according to the present disclosure, for example particulate glass compositions according to the present disclosure, may increase surface enamel microhardness. In some examples, a toothpaste, a varnish, or a prophylaxis paste according to the present disclosure may be used to increase surface enamel microhardness. In the context of the present disclosure, an increase in microhardness is in comparison to the surface enamel microhardness before any application of the presently disclosed compositions. In some examples, the surface enamel microhardness may be increased by a greater amount than the increase associated with an otherwise identical toothpaste, varnish, or prophylaxis paste that lacks the glass composition of the present disclosure.

Glass compositions according to the present disclosure, for example particulate glass compositions according to the present disclosure, may remineralize surface enamel. Without wishing to be bound by theory, the authors of the present disclosure believe that this remineralization may at least partially contribute to the increase in surface enamel microhardness.

In some examples, a toothpaste, a varnish, or a prophylaxis paste according to the present disclosure may be used to at least partially remineralize surface enamel. In the context of the present disclosure, any remineralization of the surface enamel is in comparison to the surface enamel mineralization before any application of the presently disclosed compositions. In some examples, the surface enamel may be remineralized by a greater amount than the remineralization associated with an otherwise identical toothpaste, varnish, or prophylaxis paste that lacks the glass composition of the present disclosure.

The toothpaste according to the present disclosure may be applied to the enamel of an individual, such as for a period of 30 seconds to 2 minutes, once or twice daily. In some individuals, the surface enamel microhardness may be increased after about two, three, or four days. In other individuals, the surface enamel microhardness may be increased after five days or more. In some individuals, the surface enamel may be at least partially remineralized after about two, three, or four days. In other individuals, the surface enamel may be at least partially remineralized after five days or more.

Dentin-Desensitizing Compositions

Particulate glass compositions according to the present disclosure may be formulated in a dentin-desensitizing composition that includes a water-free, orally-compatible carrier. Such dentin-desensitizing compositions according to the present disclosure are free of water since the glass composition degrades if exposed to water.

In the context of the present disclosure, “water-free” or “free of water” should be understood to mean that the dentin-desensitizing composition includes so little water that the glass composition remains capable of reducing dentin sensitivity over the expected lifespan of the product. The expected lifespan of the product refers to the longest expected time between when the dentin-desensitizing composition was produced and when the dentin-desensitizing composition was completely used up or disposed of.

The orally-compatible carrier used in the dentin-desensitizing composition may be a mouthwash, a carrier formulated to mix with additional components to form a mouthwash, or an orally-compatible viscous carrier, such as a toothpaste, a dental gel, a prophylaxis paste, a tooth varnish, a bonding agent, or a carrier that is formulated to mix with additional components to form a toothpaste. The orally-compatible viscous carrier may have a viscosity from about 100 cP at 30° C. to about 150,000 cp at 30° C.

The dentin-desensitizing composition may include a particulate glass composition according to the present disclosure in a sufficient amount that the desensitizing composition includes about 100 ppm to about 5,000 ppm of the fluoride. In some compositions according to the present disclosure, the glass composition lacks fluoride and a separate source of fluoride, such as sodium fluoride (NaF) may be added to the dentin-desensitizing composition. In the context of the present disclosure, ppm is measured in mass/mass when determining the concentration of fluoride in a desensitizing composition.

Without wishing to be bound by theory, the authors of the present disclosure believe that some glass compositions according to the present disclosure that include potassium, such as in the form of K₂O, KF, or both, may have beneficial dentin-desensitizing properties. The potassium in such glass composition may increase extracellular potassium ion concentration around nerves found in the dentin tubules. A high level of extracellular potassium ions may depolarise nerve fibre membranes and/or reduce their ability to repolarise, which ameliorates patient pain. In dentin-desensitizing compositions that include an occlusive agent and a separate potassium salt, the occlusive agent may inhibit the potassium salt from accessing the nerve, thereby reducing the ability of the separate potassium salt to ameliorate the patient pain. In contrast, some potassium-containing glass compositions according to the present disclosure may degrade while occluding the dentin tubule, and release sufficient potassium ion inside the dentin tubule that the concentration of potassium is high enough to ameliorate patient pain.

One example of a dentin-desensitizing composition according to the present disclosure is a toothpaste that includes a particulate glass composition according to the present disclosure and: an abrasive; a detergent such as sodium lauryl sulfate; a fluoride source; an antibacterial agent; a flavorant; a remineralizer; a sugar alcohol such as glycerol, sorbitol, or xylitol; another dentin desensitizing agent; a hydrophilic polymer such as polyethylene glycol; or any combination thereof. The particulate glass composition may be from about 0.5 to about 15 mass % of the toothpaste, such as about 2.5 wt % to about 7.5 wt % of the toothpaste.

One particular example of a dentin-desensitizing composition according to the present disclosure is a toothpaste that includes a particulate glass composition according to the present disclosure and: glycerin, silica, a polyethylene glycol (such as PEG 400), titanium dioxide, a carbomer, and a sweetener (such as potassium acesulfame or sodium saccharin).

Another particular example of a dentin-desensitizing composition according to the present disclosure is a toothpaste that includes a particulate glass composition according to the present disclosure and: α-carbomer, DL-limonene, glycerin, mint flavor, a polyethylene glycol (such as PEG-8), silica, titanium dioxide, sodium lauryl sulphate, and a sweetener (such as potassium acesulfame or sodium saccharin).

Another particular example of a dentin-desensitizing composition according to the present disclosure is a toothpaste that includes a particulate glass composition according to the present disclosure and: glycerin, sodium lauryl sulphate, silica (also referred to as silicon dioxide), Carbopol 940 (a crosslinked polyacrylic acid polymer, also referred to as Carbomer 940), and a flavoring agent (such as spearmint oil). The glycerin may be pure glycerol.

In a specific example, the toothpaste may contain about 85 wt % glycerol, about 1.2 wt % sodium lauryl sulphate, about 7.5 wt % silica, about 0.5 wt % Carbopol 940, about 1.0 wt % flavoring agent, and about 5.0 wt % of the particulate glass composition according to the present disclosure. The toothpaste may optionally also include sufficient sodium fluoride to result in about 1000 ppm to about 1500 ppm fluoride, such as about 0.23 wt % of NaF. The particulate glass composition may be Glass Composition #10 of Table 1A, below, sieved to obtain particles ≤25 μm.

Another example of a dentin-desensitizing composition according to the present disclosure is a carrier that includes a particulate glass composition according to the present disclosure, where the carrier is formulated to be mixed with additional components to form a toothpaste.

Yet another example of a dentin-desensitizing composition according to the present disclosure is a carrier formulated to mix with additional components to form a mouthwash. Particular examples of the carrier include a particulate glass composition according to the present disclosure and: a water-free alcohol, cetylpyridinium chloride, chlorhexidine, an essential oil, benzoic acid, a poloxamer, sodium benzoate, a flavor, a coloring, or any combination thereof. The additional component(s) that is/are mixed with the carrier to form the mouthwash may include: water, peroxide, cetylpyridinium chloride, chlorhexidine, an essential oil, alcohol, benzoic acid, a poloxamer, sodium benzoate, a flavouring, a colouring, or any combination thereof. The carrier and the additional components may be kept in separate compartments, and mixed together before the mixture is used as a mouthwash. The separate compartments may be in the form of a multi-chambered bottle, such as a bifurcated bottle.

Another example of a dentin-desensitizing composition according to the present disclosure is a prophylaxis paste (also referred to as a “prophy paste”) that includes a particulate glass composition according to the present disclosure. Particular examples of contemplated prophy pastes include a glass composition according to the present disclosure and: pumice, glycerin, diatomite (preferably fine grit), sodium silicate, methyl salicylate, monosodium phosphate, sodium carboxymethylcellulose, a sweetener (such as potassium acesulfame or sodium saccharin), a flavouring, a colouring, or any combination thereof.

Methods

Glass compositions according to the present disclosure may be synthesized by: mixing appropriate molar amounts of the starting reagents; packing the precursor blend in a platinum rhodium crucible (XRF Scientific, Perth Australia); placing the packed crucible in a furnace (Carbolite, RHF 14/3) at an initial dwelling temperature of 600 to 750° C.; holding the temperature for 60 minutes; ramping the temperature (such as at a rate of 20° C./minute) to a dwelling temperature of 1,200° C.; holding the temperature for 60 minutes; and quenching the glass melt between two stainless steel plates.

It should be understood that the specific ramp rate, times, and temperatures disclosed above could be modified, so long as the glass melts. Ramp rates from 10-20 degrees/min, and holding at the dwell temperature may remove at least some gas bubbles from the glass.

Although the resulting glass composition includes oxides, the starting reagents may include oxides, carbonates, phosphates, or any combination thereof. For example, the starting reagent may include boron oxide, calcium carbonate, sodium carbonate, and NaH₂PO₄. The calcium carbonate and sodium carbonate decompose in the furnace to release CO₂, generating their corresponding oxides. The sodium phosphate decomposes in the furnace to provide sodium and phosphorous ions within the glass oxide network. In the context of the present disclosure, it should be understood that a glass composition that includes “a phosphate source” refers to a composition that includes the decomposition products from the phosphate source; and that the mol % of the phosphate source refers to the mol % of the phosphate source starting material.

The resulting quenched glasses may be ground/milled separately within a planetary micro mill (Pulverisette 6, Fritsch, Germany) and sieved with ASTM E-11 compliant sieves (Cole Palmer, U.S.A) to obtain particles of 25 μm. Glasses may be stored under desiccating conditions in sealed storage vials.

In the context of the present disclosure, the mass loss of particulate glass compositions were measured by placing approximately 0.1 grams of the sample into a pre-weighed 15 ml Falcon tube. Ten (10) mL of TRIS buffered saline (BioUltra, Sigma Aldrich, Canada) was then pipetted into the tube. The tubes were agitated in an incubator at 120 rpm and kept at a temperature of 37° C. for the desired release period, such as for 30 minutes, 1, 3, 6, 12 or 24 h. After the specified time points elapsed, the tubes were removed from the incubator and centrifuged for 15 minutes at 1500 RCF. The supernatant was decanted into a fresh 15 mL Falcon tube. In particulate glass compositions containing a source of fluoride, the tubes containing the supernatant were sealed and stored at 4° C. until the amount of fluoride was quantified. The original 15 mL falcon tube was placed to dry at 70° C. until a constant weight was achieved to assess the residual mass of the particulate glass composition, allowing for mass loss calculations.

For particulate glass compositions containing a source of fluoride, the concentration of the fluoride released was quantified using an Accumet® AB250 pH/ion selective electrode meter equipped with an Accumet® electrode fluoride combination (Fisher Scientific, Massachusetts, USA). The standard solutions were prepared using a fluoride analytical standard specifically for ion selective electrodes (NaF, 0.1 M F, Sigma Aldrich, Canada) and calibration curves were retrieved before analysis. At the time of analysis, 1 ml of TISAB III (Fisher Scientific, Massachusetts, USA) was added into the 15 mL Falcon tube containing the supernatant at room temperature. The ion concentrations are reported as the average of n=3±SD.

Scanning electron micrograph analysis was performed using a Phenon PRoX scanning electron microscope (Thermofisher Scientific, Waltham, Mass.).

Thermal analysis of the glass samples were completed via DSC 404 F3A-0230, a high-temperature differential scanning calorimeter, with a Silicone Carbide furnace, in Pt/Rh crucibles (NETZSCH Instruments North America, Burlington Mass., USA). Approximately 0.025 grams of the samples were weighed packed in Pt/Rh crucibles. Samples were heated at a rate of 10 K/min from 20 to 900° C., with an acquisition rate of 100 pts/min under Nitrogen (Praxair, Danbury Conn., USA) protective gas at a flow rate of 50 mL/min. The Onset Temperature (Tₒ), Inflection Temperature (Tᵢ), Final Temperature (T_{f}), and Crystallization Onset Temperature (Tₚ₁) were determined with the use of Netzsch Proteus Thermal Analysis Software (VERSION 6.1.0). The Glass Transition Temperature reported in Table 4 is taken from the Onset Temperature (Tₒ) of the samples.

¹¹B magic angle spinning (MAS) NMR spectra was determined using a 16.4 T Bruker Avance NMR spectrometer (¹¹B Larmor frequency=224.67 MHz) using a 2.5 mm HX probe head operating in single resonance mode. Solid NaBH₄ was then used to calibrate the ¹¹B parameters and also utilized as an external chemical shift reference (−42.1 ppm relative to BF₃Et₂O). All samples were spun at 20 kHz MAS frequency to determine center bands and to identify spinning sidebands. For all compositions and experiments, the ¹¹B NMR was accumulated using a 0.53 μs pulse which corresponds to a 15° pulse angle in a nearly cubic environment of NaBH₄. To eliminate background noise, the spectrum of an empty rotor was acquired for each spinning speed and was subtracted from the experimental spectra.

An in vitro remineralization model was designed to be a proxy test for the ability of glass powders to promote the precipitation of mineral phases (e.g., apatite and fluoridated apatite) in the oral environment. While an ISO standard exists for the in vitro assessment of bioactivity, the ISO methods are developed for the assessment of macroscopic samples, with incubation conditions standardised to surface to volume ratios, and as such were not deemed suitable for the analysis of powders (ISO 23317:2014 “Implants for Surgery—In Vitro Evaluation for Apatite-Forming Ability of Implant Materials.”). The glass powders examined herein were fine powder (d90<30 μm), and accordingly this work is based on a protocol developed by the Technical Committee 4 of the International Commission on Glass (TCO4) to assess the bioactivity of powdered bioactive glasses and is normalized to powder weight (Macon, A. K. “A unified in vitro evaluation for apatite-forming ability of bioactive glasses and their variants.” Journal of Materials Science: Materials in Medicine, (2015) 26(2) p 115). Ground glass powders were incubated in a simulated body fluid at 37° C. Simulated Body Fluid was synthesized as per the methods and instructions published by Kokubo and Takadama (Kokubo, T. and Takadama, H. Biomaterials (2006) 27:15, pp 2907-2915). As a significant decrease in particle size can be anticipated due to the high degradation of the glasses being studied, the glass sample size was doubled from the 75 mg recommended to 125 mg, while the SBF volume was correspondingly increased from 50 mL to 100 mL. The incubated samples were removed after 30 minutes, then filtrated and dried to allow imaging to visualize mineral phase formation. Due to the intended rapid degradation of the glass powders in an aqueous environment, the TCO4 method was modified to incubate the glass powder in the simulated body fluid for 30 minutes, 3 hours, and 24 hours time points, in comparison to the 8 h, 24 h, 72 h, 1 week and 2-week timepoints used in the TCO4 method. Elemental analysis was performed using an Oxford Instruments EDX unit equipped with an 80 mm SDD with elemental mapping for 5 mins.

### Examples

The glass compositions shown in Tables 1A and 1B were all synthesized by: weighing determined amounts of the analytical grade reagents (boron oxide, calcium carbonate, sodium carbonate, magnesium oxide, sodium fluoride) (Sigma Aldrich, Canada). The individual formulations were mixed in a dry powder blender for at least 60 mins to ensure homogeneity. Each precursor blend was placed and packed in 100 mL platinum rhodium crucibles (XRF Scientific, Perth Australia). The pack crucible was then placed in a furnace (Carbolite, RHF 14/3) at an initial dwelling temperature of 600-750° C. and held for 60 minutes. The temperature was then ramped (20° C./minute) to a final dwelling temperature of 1,200° C. and held for 60 minutes. On removal, each glass melt was quenched between two stainless steel plates. The resulting quenched glasses were ground/milled separately within a planetary micro mill (Pulverisette 6, Fritsch, Germany) and sieved with ASTM E-11 compliant sieves (Cole Palmer, U.S.A) to obtain particles of 25 μm.

TABLE 1A
Exemplary glass compositions according to the
present disclosure (components listed in mol %)

| Composition | | | | | |
|---|---|---|---|---|---|
| No. | B₂O₃ | Na₂O | CaO | MgO | NaF |
| 1 | 43.5 | 10.3 | 15.4 | 20.6 | 10.3 |
| 2 | 39.5 | 11.0 | 16.5 | 22.0 | 11.0 |
| 3 | 35.5 | 11.7 | 17.5 | 23.5 | 11.7 |
| 4 | 31.5 | 12.5 | 18.6 | 24.9 | 12.5 |
| 5 | 32.5 | 13.9 | 20.8 | 27.8 | 13.9 |
| 6 | 27.5 | 13.2 | 19.7 | 26.4 | 13.2 |
| 7 | 45.0 | 20.0 | 15.0 | 20.0 | — |
| 8 | 45.0 | 17.5 | 17.5 | 20.0 | — |
| 9 | 45.0 | 15.5 | 19.5 | 20.0 | — |
| 10 | 43.0 | 15.6 | 20.7 | 20.7 | — |

TABLE 1B
Exemplary glass compositions according to the present disclosure
(components listed in mol %), as well as two of their
bulk properties (% crystallinity and density)

| Composition | | | | | | % | density |
|---|---|---|---|---|---|---|---|
| No. | B₂O₃ | Na₂O | K₂O | CaO | MgO | crystallinity | (g/cm³) |
| 1.02 | 41.57 | 5.00 | 0.00 | 24.86 | 28.57 | 1.8 | 2.72 |
| 1.08 | 45.00 | 5.00 | 10.00 | 5.00 | 35.00 | 1.0 | 2.44 |
| 1.09 | 45.00 | 21.20 | 0.00 | 21.85 | 11.95 | 3.9 | 2.57 |
| 2.02 | 39.53 | 19.33 | 0.00 | 16.14 | 25.00 | 1.6 | 2.56 |
| 2.03 | 45.00 | 25.00 | 3.76 | 5.00 | 21.24 | 1.4 | 2.32 |
| 2.04 | 42.31 | 5.00 | 4.69 | 25.00 | 23.00 | 1.5 | 2.59 |
| 2.06 | 31.00 | 7.73 | 17.58 | 19.78 | 23.91 | 2.2 | 2.52 |
| 2.07 | 45.00 | 5.00 | 17.52 | 7.48 | 25.00 | 1.2 | 2.35 |
| 2.14 | 45.00 | 15.07 | 10.04 | 16.04 | 13.86 | 1.6 | 2.47 |
| 2.16 | 45.00 | 9.78 | 6.34 | 13.91 | 24.98 | 1.6 | 2.45 |
| 2.17 | 35.35 | 15.09 | 12.68 | 11.88 | 25.00 | 0.4 | 2.46 |
| 2.22 | 43.12 | 16.30 | 0.00 | 25.00 | 15.58 | 1.4 | 2.57 |
| 2.23 | 29.00 | 25.00 | 14.41 | 6.59 | 25.00 | 0.8 | 2.39 |
| 2.26 | 38.60 | 17.00 | 12.00 | 17.00 | 15.40 | 0.7 | 2.46 |
| 3.01 | 45.00 | 18.00 | 15.00 | 9.85 | 12.15 | 1.2 | 2.36 |
| 3.03 | 45.00 | 18.00 | 3.04 | 22.00 | 11.96 | 1.6 | 2.51 |
| 3.04 | 29.00 | 18.00 | 15.00 | 16.00 | 22.00 | 3.2 | 2.50 |
| 3.05 | 45.00 | 18.00 | 0.00 | 15.00 | 22.00 | 1.2 | 2.45 |
| 3.06 | 45.00 | 13.00 | 7.44 | 12.55 | 22.00 | 1.5 | 2.41 |
| 3.07 | 45.00 | 11.00 | 0.00 | 22.00 | 22.00 | 1.7 | 2.53 |
| 3.08 | 40.70 | 14.45 | 10.95 | 17.45 | 16.45 | 1.4 | 2.40 |
| 3.09 | 36.38 | 13.85 | 15.00 | 12.77 | 22.00 | 1.5 | 2.36 |
| 3.11 | 45.00 | 18.00 | 10.00 | 5.00 | 22.00 | 1.3 | 2.30 |
| 3.12 | 34.86 | 18.00 | 3.14 | 22.00 | 22.00 | 2.1 | 2.50 |
| 3.14 | 36.00 | 5.00 | 15.00 | 22.00 | 22.00 | 1.1 | 2.47 |
| 3.16 | 45.00 | 5.77 | 15.00 | 12.23 | 22.00 | 1.6 | 2.41 |
| 3.19 | 45.00 | 5.00 | 6.02 | 21.98 | 22.00 | 1.5 | 2.51 |
| 3.20 | 42.08 | 13.34 | 15.00 | 20.50 | 9.07 | 1.4 | 2.42 |
| 3.21 | 45.00 | 5.00 | 15.00 | 22.00 | 13.00 | 1.4 | 2.43 |
| 3.23 | 40.00 | 18.00 | 15.00 | 5.00 | 22.00 | 0.9 | 2.68 |
| 3.24 | 34.86 | 18.00 | 3.14 | 22.00 | 22.00 | 1.3 | 2.53 |

Some of the particles of the exemplary glasses of Table 1A were evaluated for mass loss using the method discussed above. The percent mass loss after 1 and 24 hours are shown in Table 2.

TABLE 2
Mass loss at 1 and 24 hours.

| Composition | Mass Loss (%) | Mass Loss (%) |
|---|---|---|
| No. | at 1 hour | at 24 hours |
| 7 | 73.8173 | 88.4176 |
| 8 | 75.8858 | 84.1346 |
| 9 | 58.3940 | 82.0945 |
| 10 | 60.5955 | 63.7153 |

Some of the particles of the exemplary glasses of Table 1B were evaluated for mass loss using the method discuss above. The percent mass loss after 30 minutes is shown in Table 3.

TABLE 3
Percent mass loss after 30 minutes

| Composition | Mass loss (%) |
|---|---|
| No. | at 30 minutes |
| 2.02 | 47 |
| 2.03 | 71 |
| 2.04 | 64 |
| 2.06 | 59 |
| 2.07 | 83 |
| 2.14 | 76 |
| 2.16 | 73 |
| 2.17 | 57 |
| 2.22 | 65 |
| 2.23 | 57 |
| 2.26 | 69 |
| 3.01 | 76 |
| 3.03 | 77 |
| 3.04 | 72 |
| 3.05 | 71 |
| 3.06 | 76 |
| 3.07 | 59 |
| 3.08 | 59 |
| 3.09 | 60 |
| 3.11 | 87 |
| 3.12 | 46 |
| 3.14 | 50 |
| 3.16 | 90 |
| 3.19 | 66 |
| 3.20 | 77 |
| 3.21 | 83 |
| 3.23 | 63 |
| 3.24 | 37 |

The density of the glass powders were measured using an AccuPyc 1340 helium pycnometer (Micromeritics, USA) equipped with a 1 cm³ insert. Prior to use, a traceable volume standard was used to calibrate the pycnometer. For glass powder analysis, the insert was packed with approximately 1 g of glass powder. Each measurement is calculated from the mean of 10 readings.

The percentage of amorphous phase of the samples was assessed using a D2 Phaser X-ray diffractometer, with a Cu source, and a Lynxeye linear array detector (Bruker AXS Inc, Maddison Wis., USA). Diffraction spectra of finely ground samples were collected between 2 theta angles for 10 to 60 degrees, with a step size of 0.02 degrees and a dwell time of 2 seconds. The relative volume of amorphous material was calculated by fitting a background curve to the amorphous halo, and calculating the relative intensity of the background corrected reduced area to the uncorrected global area. The percent amorphous phase is related to the percent crystallinity by the equation (% crystallinity)+(% amorphous phase)=100.

The particles of the exemplary glasses of Table 1A had the following bulk properties:

TABLE 4
Bulk properties for some exemplary glasses

| Composition | Density | | Glass Transition |
|---|---|---|---|
| No. | (g/cm³) | % Crystallinity | Temp (° C.) |
| 1 | 2.5355 | 1.8 | 472.8 |
| 2 | 2.5616 | 2.5 | 449.7 |
| 3 | 2.5842 | 2.1 | 418.4 |
| 4 | 2.453 | 2.9 | 394.9 |
| 5 | 2.6973 | 13.3 | 353.2 |
| 6 | 2.6365 | 5.2 | 370.5 |
| 7 | 2.5040 | 6.3 | 470.3 |
| 8 | 2.5168 | 4.6 | 484.2 |
| 9 | 2.5357 | 3.6 | 496.1 |
| 10 | 2.582 | 7.3 | 473.7 |

Table 1B contains compositions of a design space defined by the following table, where the units are in mol %.

TABLE 5
Mixture design constraints for a design space.

| Comp. | Name | Type | Min. | Max. | Coded low | Coded high | Mean | Std. Dev |
|---|---|---|---|---|---|---|---|---|
| A | B₂O₃ | mixture | 29 | 45 | +0 ↔ 29 | +0.266667 ↔ 45 | 40.71 | 5.40 |
| B | K₂O | mixture | 0 | 15 | +0 ↔ 0 | +0.25 ↔ 15 | 10.36 | 5.57 |
| C | CaO | mixture | 5 | 22 | +0 ↔ 5 | +0.283333 ↔ 22 | 17.21 | 5.68 |
| D | MgO | mixture | 1 | 22 | +0 ↔ 1 | +0.35 ↔ 22 | 17.45 | 5.81 |
| E | NaO | mixture | 5 | 18 | +0 ↔ 5 | +0.216667 ↔ 18 | 14.26 | 4.71 |

| | |
|---|---|
| Total = 100 | L_Pseudo Coding |

The results of the tested compositions, within a design space, provided the following equations, which may allow for the relative comparison of different compositions and/or which may be useful to identify trends associated with different components of the compositions. While experimental and modeling error prevents absolute prediction of glass properties, the equations may be used to guide and refine glass composition design. When used together, these models may help suggest which factors may be traded off in the tailoring of multi-component compositions within the tested composition space. In the following equations, the values for the listed components are in percentages (not fractions or decimals). For example, 50 mol % of B₂O₃ would be “50” (and not “0.5”).

The crystallinity of a melt may be generally predicted under the tested quench conditions using the following formula:

Crystallinity=−7.21994*[B₂O₃]+10.5814*[K₂O]+13.6798*[CaO]+16.9661*[MgO]+4.75849*[NaO]−35.849*[B₂O₃][K₂O]−45.4598*[B₂O₃][CaO]−66.4434*[K₂O][MgO]−66.849*[CaO][MgO]−72.7346*[MgO][NaO].

The density of a glass may be generally predicted using the following formula:

ρ=2.14644*[B₂O₃]+2.24491*[K₂O]+2.92911*[CaO]+2.43832*[MgO]+2.42776*[NaO].

Glass densities from about 1.3 g/cm³ to about 2.2 g/cm³ may particularly useful in non-aqueous oral care formulations. Glycerol and silica, which are the primary liquid and solid components of a non-aqueous toothpaste, have densities of 1.3 and 2.2 g/cm³, respectively.

The NMR B3 chemical shift (ppm) may be generally predicted using the following formula:

ppm=6.74673*[B₂O₃]+3.33975*[K₂O]+7.20888*[CaO]+10.1749*[MgO]+4.01478*[NaO]−11.8899*[B₂O₃][K₂O]−25.2187*[B₂O₃][CaO]−25.023*[B₂O₃][MgO]−12.4656*[B₂O₃][NaO]−12.5781*[K₂O][MgO]−18.8676*[CaO][MgO]−19.0726*[MgO][NaO].

NMR provides a tool to probe the local environment of the ¹¹B atoms in the glass. The percentage of the networks configured as B3 (trigonal) versus B4 (tetrahedral) co-ordinated B can be determined using NMR. Unexpectedly, the authors of the present disclosure determined that the influence (from the coefficients) of alkali and alkaline earth elements has a similar effect on the network configuration. The ratios provided in this data, support in addition to the compositional chemistry, the mechanistic basis for degradation.

The equation related to percent of mass loss after 30 minutes under the tested conditions is:

1189.44*[B₂O₃]−87.7623*[K₂O]−62.9762*[CaO]+375.296*[MgO]−80.86*[NaO]−982.106*[B₂O₃][K₂O]−1169.24*[B₂O₃][CaO]−2192.55*[B₂O₃][MgO]−1040.75*[B₂O₃][NaO]+485.18*[K₂O][CaO]−139.18*[K₂O][MgO]+283.37*[K₂O][NaO]−460.87*[CaO][MgO]+475.861*[CaO][NaO]−304.428*[MgO][NaO].

Six exemplary glass compositions were tested for their ability to remineralize surface enamel. The tested compositions were: composition 10, as identified in Table 1A; and compositions 3.01, 3.04, 3.06, 3.20 and 3.24, as identified in Table 1B.

The results of the remineralization are illustrated in Tables 6, 7 and 8, below.

TABLE 6
Atomic %, average of three replicates, ±SD, time = 0 hours (control).

| Comp. | | | | | | | | | Ratio |
|---|---|---|---|---|---|---|---|---|---|
| No. | B | O | Na | Mg | K | Ca | C | P | Ca:P |
| 10 | 38.2 ± 1.5 | 49.3 ± 2.7 | 5.6 ± 0.5 | 3.7 ± 0.4 | nd | 3.3 ± 0.5 | nd | nd | n/a |
| 3.01 | 24.9 ± 0.4 | 50.1 ± 2.1 | 5.8 ± 0.6 | 1.8 ± 0.2 | 5.1 ± 1.0 | 1.7 ± 0.3 | 10.6 ± 2.0 | nd | n/a |
| 3.04 | 20.3 ± 2.0 | 47.2 ± 0.9 | 7.0 ± 0.1 | 3.8 ± 0.1 | 6.2 ± 1.2 | 3.1 ± 0.5 | 12.5 ± 0.8 | nd | n/a |
| 3.06 | 24.7 ± 2.5 | 43.2 ± 3.2 | 4.2 ± 0.5 | 3.3 ± 0.5 | 2.9 ± 0.7 | 2.3 ± 0.5 | 19.3 ± 3.3 | nd | n/a |
| 3.20 | 29.9 ± 2.0 | 47.9 ± 2.0 | 4.1 ± 0.4 | 1.3 ± 0.2 | 4.2 ± 1.2 | 3.0 ± 0.8 | 9.6 ± 1.3 | nd | n/a |
| 3.24 | 20.1 ± 3.0 | 50.1 ± 0.8 | 7.0 ± 0.2 | 3.9 ± 0.2 | 1.7 ± 0.7 | 6.3 ± 2.6 | 11.1 ± 1.5 | nd | n/a |

“nd” means none detected.

TABLE 7
Atomic %, average of three replicates, ±SD, time = 30 minutes.

| Comp. | | | | | | | | | Ratio |
|---|---|---|---|---|---|---|---|---|---|
| No. | B | O | Na | Mg | K | Ca | C | P | Ca:P |
| 10 | nd | 69.4 ± 6.7 | 2.2 ± 3.5 | 2.7 ± 1.9 | nd | 9.7 ± 1.6 | 9.1 ± 9.1 | 6.9 ± 3.3 | 1.40 |
| 3.01 | nd | 74.1 ± 3.8 | 0.3 ± 0.4 | 2.0 ± 0.1 | nd | 13.3 ± 2.2 | nd | 10.3 ± 1.6 | 1.29 |
| 3.04 | nd | 63.6 ± 16.9 | 0.2 ± 0.1 | 2.7 ± 0.3 | nd | 19.6 ± 10.5 | nd | 13.9 ± 6.3 | 1.41 |
| 3.06 | nd | 75.6 ± 1.3 | 0.1 ± 0.02 | 2.2 ± 0.09 | nd | 12.3 ± 1.0 | nd | 9.7 ± 0.4 | 1.26 |
| 3.20 | nd | 77.3 ± 6.5 | 0.3 ± 0.5 | 1.4 ± 0.1 | 0.3 ± 0.5 | 12.6 ± 3.6 | nd | 8.2 ± 4.0 | 1.53 |
| 3.24 | nd | 79.3 ± 3.3 | 1.9 ± 1.6 | 4.1 ± 2.2 | 0.3 ± 0.1 | 9.5 ± 3.4 | nd | 5.0 ± 3.8 | 1.90 |

“nd” means none detected.

TABLE 8
Atomic %, average of three replicates, ±SD, time = 24 hours.

| Comp. | | | | | | | | | Ratio |
|---|---|---|---|---|---|---|---|---|---|
| No. | B | O | Na | Mg | K | Ca | C | P | Ca:P |
| 10 | nd | 78.7 ± 2.8 | nd | 1.8 ± 0.08 | nd | 10.4 ± 1.5 | nd | 9.2 ± 1.2 | 1.13 |
| 3.01 | nd | 76.0 ± 1.9 | nd | 2.0 ± 0.1 | nd | 12.0 ± 1.0 | nd | 10.0 ± 0.9 | 1.20 |
| 3.04 | nd | 78.5 ± 2.3 | nd | 2.1 ± 0.2 | nd | 10.9 ± 1.1 | nd | 8.6 ± 0.9 | 1.26 |
| 3.06 | nd | 79.1 ± 1.9 | nd | 2.2 ± 0.1 | nd | 10.0 ± 1.0 | nd | 8.7 ± 0.8 | 1.15 |
| 3.20 | nd | 78.1 ± 0.7 | 0.1 ± 0.1 | 1.2 ± 0.0 | nd | 11.7 ± 0.3 | nd | 8.9 ± 0.3 | 1.31 |
| 3.24 | nd | 77.4 ± 1.1 | nd | 1.8 ± 0.0 | nd | 11.4 ± 0.6 | nd | 9.4 ± 0.5 | 1.21 |

“nd” means none detected.

In addition, the remineralization results for Compound 10 were measured at 3 hours. The atomic percentages, as an average of three replicates (±SD), were: B: not detected; O: 75.7±2.3; Na: 0.1±0.006; Mg: 1.9±0.09; K: not detected; Ca: 12.1±1.3; C: not detected; and P: 10.3±0.9.

Calcium (Ca) and phosphorous (P) are the building blocks of amorphous calcium phosphates and apatites, which act to remineralize teeth. Identifying these elements at the surface of a glass incubated in SBF indicates the mineralization capacity of that glass. The literature typically says mineralization occurs over hours (typically 24 hours), days, or weeks. The tested formulations, which are deficient in P, show Ca and P containing precipitates after only 30 mins. The results in Tables 6, 7 and 8 illustrate that, at time=0 hours, no phosphorous was detected on the surface of the glass particles. The detected carbon (“C”) reflects surface contamination that occurred during sample preparation. At time=24 hours, phosphorous was detected in a ratio with calcium ranging from 1.13:1 to 1.31:1 (Ca:P), which approaches the approximate 1.6 ratio of calcium to phosphorous present in apatite.

An exemplary toothpaste (“5% SIP-FF+NaF”) was prepared using Glass Composition No. 10 (i.e. a glass composition consisting of 43.0 mol % of B₂O₃, 20.7 mol % MgO, 20.7 mol % CaO, and 15.6 mol % Na₂O) according to the following table:

TABLE 9
Exemplary toothpaste formulation “5% SIP-FF + NaF”.

| Ingredient | Amount (wt %) |
|---|---|
| Glycerol | 84.57 |
| Sodium Lauryl Sulfate | 1.20 |
| Silicon Dioxide | 7.50 |
| Glass Composition No. 10 | 5.00 |
| (particle size ≤25 micron) | |
| Carbopol 940 | 0.50 |
| Flavour (Spearmint oil) | 1.00 |
| Sodium Fluoride (NaF) | 0.23 |
| | (1040 ppm fluoride) |

The glass particles were sieved to collect ≤25-micron particles. Particle size analysis confirmed that the powdered particles were appropriately sized to occlude dentin tubules, which typically have diameters from 1 to 5 μm. The mean particle size distribution of the glass was D10=6.46 μm, D50=16.6 μm, and D90=33.0 μm, where Dₓ is the diameter where X % of the distribution has a diameter smaller than the D.

The exemplary toothpaste 5% SIP-FF+NaF was tested in single-time point, and multi-time point, dentin occlusion studies, as well as a single time point hydraulic conductance study.

Single-time point dentin occlusion study. The 5% SIP-FF+NaF toothpaste was compared against commercial toothpaste products: (Control Article #1) Sensodyne® Repair and Protect with NOVAMIN® (5% Novamin and 1040 ppm fluoride as sodium fluoride), and (Control Article #2) Colgate® Sensitive PRO-Relief™ (8% Arginine, 35% Calcium carbonate 1320 ppm fluoride as sodium monofluorphosphate) in a single-time point dentin occlusion study.

Analysis of dentin samples treated twice daily using both simulated brushing for 2 minutes, and direct application of a pea-sized amount to an area of sensitivity using a clean finger, provides a measurement of the degree of dentin tubule blockage by the subject toothpastes after one day of treatment. The degree of dentin tubule blockage is commonly understood in the art to be an indirect measure of the ability to reduce dentin hypersensitivity; that is, as the level of occlusion increases, the dentin fluid flow will decrease thereby resulting in decreased sensation of pain. The reduction of dentin fluid flow reduces sensitivity and the precipitation of fluoridated apatites provides a barrier for fast relief. Fluoridated apatites, which help prevent tooth decay or dental caries, may be formed in the presence of fluoride ions in solution, which are incorporated into the mineral.

Human dentin samples (about 1.0 to about 1.5 mm thick) were prepared from the crowns of caries-free unrestored molars, perpendicular to the long axis of the root, using a diamond disc saw. Each section was etched for 2 minutes with 10% citric acid, followed by water rinsing for 60 seconds, sonification for 2 minutes in deionised water, and further rinsed for 60 seconds in water. Each section was placed into a mould and covered with acrylic resin. Once hardened, the dentin face was polished to a mirror finish. Following a rinse with deionised water, the surface was etched, sonicated and rinsed again. Sample integrity, tubule density and patency were verified under scanning electron microscopy (SEM) using a Phenon PRoX scanning electron microscope (Thermofisher Scientific, Waltham, Mass.).

Artificial saliva (30 mM potassium chloride, 13 mM sodium chloride, 10 mM potassium dihydrogen orthophosphate, 3 mM calcium chloride dehydrate, 0.22% w/w Type II Porcine Stomach Mucin, and 0.02% w/w sodium azide) was prepared. The dentin samples were immersed in the artificial saliva for at least 60 minutes at 37° C. prior to treatment with the toothpastes.

For brushing application, 0.67 g of toothpaste was applied to the dentin sample using an oscillating Oral-B Precision toothbrush for 10 seconds. For direct application, 0.25 g of toothpaste was applied to the dentin sample using light pressure and a gloved finger for 10 seconds in circular motions. The dentin sample treatment and application conditions are summarized below in Table 10:

TABLE 10

| | Brushing | | Direct | |
|---|---|---|---|---|
| Method of treatment and application | Application | | Application | |
| Number of Treatment Days | 1 | | 1 | |
| # Treatments/Day | 2 | | 2 | |
| # Replicate Samples | 4 | | 4 | |
| Treatment Quantity | 0.67 | g | 0.25 | g |
| Treatment Duration | 10 | sec. | 10 | sec. |

| | | |
|---|---|---|
| Storage between treatments | Artificial | Artificial |
| | saliva | saliva |

For both application methods, the samples were rinsed for 30 seconds with deionised water following application to remove visible signs of the toothpaste, then stored in artificial saliva for at least one hour before the application cycle was repeated to simulate twice daily use. Following the second application, samples were treated again in simulated saliva for 60 seconds before drying and preparation for SEM imaging.

Treated dentin samples with gold sputter coating were imaged using an Phenon ProX Scanning Electron Microscope, with 3 images collected at ×3000 magnification for each sample. Each SEM image was assessed by two double blinded assessors for the extent of denting occlusion based on a five point categorical scale, using the following grading classification:

1. Occluded

2. Mostly occluded

3. Equal

4. Mostly unoccluded

5. Unoccluded

Data analysis was performed using Minitab 18 software. All treatment groups were assessed to provide descriptive statistics of group mean, standard deviation, minimum, maximum, and number of replicates. All data sets were then tested for normalcy. For data sets which passed the assumption of normalcy, 2-sample t-tests were used to make pairwise comparisons between data sets. For pairings where one of more data sets failed to meet the assumption of normalcy, a Mann-Whitney test was used to make pairwise statistical comparisons. All statistical tests were performed at a 0.05 significance level.

Initial performance data supports that the 5% SIP-FF+NaF toothpaste is effective and has the ability to partially occlude dentin tubules. The mean occlusion scores are:

TABLE 11

| | Brushing | |
|---|---|---|
| | Application | Direct Application |
| | Mean Occlusion | Mean Occlusion |
| Toothpaste | Score¹ | Score¹ |
| 5% SIP-FF + NaF | 2.7 ± 0.8 | 3.7 ± 0.8 |
| Colgate ® Sensitive | 3.8 ± 0.3 | 3.8 ± 0.5 |
| PRO-Relief ™ | | |
| Sensodyne ® Repair and | 3.9 ± 0.2 | 4.3 ± 0.4 |
| Protect with NOVAMIN ® | | |

¹Categorical occlusion grading where 1 = Occluded, 2 = Mostly occluded, 3 = Equally occluded and unocculded, 4 = Mostly unoccluded, and 5 = Unoccluded.

SEM images of dentin tubules treated with the 5% SIP-FF+NaF toothpaste show tubule occlusion both by larger undegraded particles retained within the dentin tubule or on the dentin surface, as well as the development of smaller mineral deposits within the dentin tubule.

In addition to intratubular occlusion, formation of a layer on the exposed dentin surface may obstruct the tubules. As the glass composition degrades, the rate of which is influenced by particle size, beneficial ions are released to promote the formation of apatites, including fluoride containing apatites.

Sensodyne® Repair and Protect with NOVAMIN® was the worst performing toothpaste at occluding dentin tubules for both the brushing and direct application. Marketing literature claims that Sensodyne® Repair and Protect with NOVAMIN® “starts working from week 1” supporting that it may exert more of a build-up effect over several days rather than an immediate benefit as demonstrated here by Sensi-IP®. Independent in vitro studies conducted by the Technical Committee 4 of the International Commission on Glass (TCO4) on the original bioactive glass composition 45S5, which is the basis of the Novamin Technology, found that it took 24 hours to begin to see effects of surface reaction in vitro (J Mater Sci: Mater Med 2015).

Multi-time point dentin occlusion study. The 5% SIP-FF+NaF toothpaste described above was also compared against commercial toothpaste products: (Control Article #1) Sensodyne® Repair and Protect with NOVAMIN® (5% Novamin and 1040 ppm fluoride as sodium fluoride), and (Control Article #2) Colgate® Sensitive PRO-Relief™ (8% Arginine, 35% Calcium carbonate 1320 ppm fluoride as sodium monofluorphosphate) in a multi-time point dentin occlusion study over 5 simulated treatment days.

Analysis of dentin samples treated twice daily using simulated brushing for 2 minutes for one to five days provides a measurement of the degree of dentin tubule blockage by the subject toothpastes over several days. The degree of dentin tubule blockage is commonly understood in the art to be an indirect measure of the ability to reduce dentin hypersensitivity; that is, as the level of occlusion increases, the dentin fluid flow will decrease thereby resulting in decreased sensation of pain.

Human dentin samples were prepared in the same manner as in the single-time point dentin occlusion study, discussed above.

Artificial saliva (30 mM potassium chloride, 13 mM sodium chloride, 10 mM potassium dihydrogen orthophosphate, 3 mM calcium chloride dehydrate, 0.22% w/w Type II Porcine Stomach Mucin, and 0.02% w/w sodium azide) was prepared. The dentin samples were immersed in the artificial saliva for at least 60 minutes at 37° C. prior to the first treatment with the toothpastes.

Samples were treated with the toothpastes (Table 12) twice daily by brushing with 0.67 g of toothpaste with an oscillating toothbrush for 10 seconds.

TABLE 12

| | | # Replicates/ | |
|---|---|---|---|
| | | Treatment | Treatment |
| Treatment | Treatment Description | Group | Groups |
| Test Article | 5% SIP-FF + NaF Paste | 4 | 1, 2, 3, |
| Control Article #1 | Sensodyne ® Repair and | 4 | 4, and |
| | Protect with NOVAMIN ® | | 5-Days |
| Control Article #2 | Colgate ® Sensitive | 4 | |
| | PRO-Relief ™ | | |

The samples were treated for one to five days as outlined in Table 13. Samples were rinsed for 30 seconds with deionised water following application to remove visible signs of the toothpaste, then stored in artificial saliva for at least one hour before the application cycle was repeated to simulate twice daily use. Following the twice-daily application, samples were soaked in simulated saliva for 3 hours before being transferred into dampened tissue until the next treatment timepoint.

TABLE 13

| Treatment Group | 1 | | 2 | | 3 | | 4 | | 5 | |
|---|---|---|---|---|---|---|---|---|---|---|
| Number of | 1 | | 2 | | 3 | | 4 | | 5 | |
| Treatment Days | | | | | | | | | | |
| # Treatments/Day | 2 | | 2 | | 2 | | 2 | | 2 | |
| Treatment Quantity | 0.67 | g | 0.67 | g | 0.67 | g | 0.67 | g | 0.67 | g |
| Treatment Duration | 10 | sec. | 10 | sec. | 10 | sec. | 10 | sec. | 10 | sec. |

| | | | | | |
|---|---|---|---|---|---|
| Storage between | Artificial | Artificial | Artificial | Artificial | Artificial |
| treatments | saliva | saliva | saliva | saliva | saliva |
| Total # | 2 | 4 | 6 | 8 | 10 |
| Treatments/Time- | | | | | |
| Point | | | | | |
| Total # | 12 | 12 | 12 | 12 | 12 |
| Samples/Treatment | | | | | |

Treated dentin samples with gold sputter coating were imaged using an Phenon ProX Scanning Electron Microscope, with 3 images collected at ×3000 magnification for each sample. Each SEM image was assessed by two double blinded assessors for the extent of denting occlusion based on a five point categorical scale, using the following grading classification:

1. Occluded

2. Mostly occluded

3. Equal

4. Mostly unoccluded

5. Unoccluded

Data analysis was performed using Minitab 18 software. All treatment groups were assessed to provide descriptive statistics of group mean, standard deviation, minimum, maximum, and number of replicates. All data sets were then tested for normalcy. For data sets which passed the assumption of normalcy, 2-sample t-tests were used to make pairwise comparisons between data sets. For pairings where one of more data sets failed to meet the assumption of normalcy, a Mann-Whitney test was used to make pairwise statistical comparisons. All statistical tests were performed at a 0.05 significance level.

Initial performance data supports that the 5% SIP-FF+NaF toothpaste is effective and has the ability to partially occlude dentin tubules. The mean occlusion scores are:

TABLE 14
Mean occlusion scores (+/−SD) for each toothpaste
after 1, 2, 3 and 4 days of application (ranging
from 1 for fully occluded to 5 for unoccluded).

| Toothpaste | Day 1 | Day 2 | Day 3 | Day 4 | Day 5 |
|---|---|---|---|---|---|
| 5% SIP-FF + NaF | 3.8 | 2.7 | 2.5 | 2.0 | 1.8 |
| | (+/−0.6) | (+/−0.7) | (+/−1.1) | (+/−0.7) | (+/−0.6) |
| Colgate ® Sensitive | 4.4 | 4.0 | 3.7 | 3.2 | 3.1 |
| PRO-Relief ™ | (+/−0.4) | (+/−0.7) | (+/−0.4) | (+/−0.6) | (+/−0.5) |
| Sensodyne ® Repair | 4.0 | 4.0 | 3.2 | 3.5 | 3.9 |
| and Protect with | (+/−0.6) | (+/−0.1) | (+/−0.8) | (+/−0.5) | (+/−0.8) |
| NOVAMIN ® | | | | | |

Full occlusion (represented by occlusion scores of 1) was achieved by some Sensi-IP® toothpaste treated dentin samples after 3 days of application of 5% SIP-FF+NaF toothpaste. No other toothpastes achieved an occlusion score of 1 for any of the samples treated over the treatment period.

Sensodyne® Repair and Protect with NOVAMIN® and Colgate® Sensitive PRO-Relief™ demonstrated equivalent performance over all timepoints and were inferior to the 5% SIP-FF+NaF toothpaste for providing visual occlusion.

Surface Microhardness. Enamel blocks shaped to approximately 4 by 4 mm were sliced from labial bovine incisors, lapped and polished to a grit of 0.04 μm. One corner was abraded off to allow for sample orientation, and samples were stored, refrigerated, and dampened with 0.1% thymol until use.

Baseline surface microhardness measurements were assessed using the Wilson Tukon 1202 microhardness tester. A series of 8 indentations were made at 100 μm spacing, using a 50 g load and 10 second dwell time. Measurement of indent size was performed using an 50× objective. Samples were accepted into the study with an inclusion criterion of a SMH of ≥250 HK, and standard deviation of ≤20 HK. Following baseline assessment, an initial demineralization challenge was applied by soaking the samples in 8 ml of demineralization solution per block at 37° C. for 60 minutes, followed by a deionized water rinse. Surface microhardness measurements were taken for each enamel block both before demineralization as a quality check for inclusion in the study, after initial demineralization treatment, and following pH cycling treatment:

TABLE 15

| Step | Solution | Volume | | Duration | |
|---|---|---|---|---|---|
| 1 | Toothpaste slurry | 5 | ml | 2 | minutes |
| 2 | Remineralizing solution | 20 | ml | 58 | minutes |
| 3 | Toothpaste slurry | 5 | ml | 2 | minutes |
| 4 | Remineralizing solution | 20 | ml | 58 | minutes |
| 5 | Demineralizing solution | 20 | ml | 60 | minutes |
| 6 | Remineralizing solution | 20 | ml | 120 | minutes |
| 7 | Toothpaste Slurry | 5 | ml | 2 | minutes |
| 8 | Remineralizing solution | 20 | ml | 58 | minutes |
| 9 | Toothpaste Slurry | 5 | ml | 2 | minutes |

| | | | | |
|---|---|---|---|---|
| 10 | Remineralizing solution | 20 | ml | Overnight |

| | |
|---|---|
| 11 | Repeat Steps 1-10 four more times (5 days total) |

A negative control paste was used for comparison, consisting of the equivalent toothpaste chassis without the addition of SIP-FF, along with a positive control which consisted of the equivalent chassis, without SIP-FF, and the addition of 1040 ppm F as NaF.

Surface microhardness (SMH) was analyzed using a series of 8 indents made at 100 μm spacing using a 50 g load and 10 second dwell time. Measurements of the indents was taken using a 50× objective, and hardness was expressed as Hardness Knoop.

Surface microhardness recovery (SMHR) was calculated using the following equation:

$${}_{}^{c} {/}_{o}^{} SMHR = 100 \times [ \frac{SMH final - SMH Demineralized}{SMH Baseline - SMH Demineralized} ]$$

All statistical analysis was performed using Minitab 18 software. For each experiment, summary statistics were generated for each treatment group and timepoint (n, mean, standard deviation). All data sets were tested for normality using the Anderson-Darling test. Pairwise comparison was performed between treatment groups for each experiment and timepoint. For the enamel surface microhardness experiments, all data sets satisfied the assumptions criteria, and one-way ANOVA was used to compare experimental results. For the visual occlusion experiment, and fluoride uptake tests, 2-sample T tests were used to make pairwise comparisons between occlusion scores when assumptions of normality could be met, and a Mann-Whitney test was used to make comparisons when one or more of the pair failed the normality test. All statistical tests were performed at a 0.05 significance level.

TABLE 16
Mean percentage surface microhardness recovery (+/−) SD
for each toothpaste after 5 days of pH cycling treatment.

| | | | | Statistical |
|---|---|---|---|---|
| | Fluoride | | Mean percentage | comparison |
| Toothpaste | content | | of SMHR | groupings |
| 5% SIP-FF + NaF | 1040 | ppm | 58.9 ± 14.6% | A |
| 5% SIP-FF | 0 | ppm | 5.8 ± 18.8% | B, C |
| Positive Control | 1040 | ppm | 23.2 ± 24.9% | B |
| (NaF control) | | | | |
| Negative Control | 0 | ppm | −6.3 ± 14.2% | C |
| (blank control) | | | | |

In the preceding description, for purposes of explanation, numerous details are set forth in order to provide a thorough understanding of the examples. However, it will be apparent to one skilled in the art that these specific details are not required. Accordingly, what has been described is merely illustrative of the application of the described examples and numerous modifications and variations are possible in light of the above teachings.

Since the above description provides examples, it will be appreciated that modifications and variations can be effected to the particular examples by those of skill in the art. Accordingly, the scope of the claims should not be limited by the particular examples set forth herein, but should be construed in a manner consistent with the specification as a whole.

1. A glass composition comprising:
from about 20 mol % to 45 mol % of B₂O₃;
from about 10 mol % to about 80 mol % of one or more glass components selected from the group consisting of CaO and MgO;
optionally one or more glass components selected from the group consisting of Na₂O, K₂O, and a phosphate source; and
optionally up to about 45 mol % of CaF₂, SnF₂, NaF, KF, Na₂PO₃F, or a combination thereof;
wherein the B₂O₃ and the phosphate source total about 60 mol % or less; and
wherein the composition comprises less than 0.1 mol % CdO.

2. The glass composition according to claim 1, wherein the phosphate source is additionally less than about 35 mol %, less than about 30 mol %, less than about 25 mol %, less than about 20 mol %, less than about 15 mol %, less than about 10 mol %, or less than about 5 mol % of the glass composition.

3. The glass composition according to claim 1, wherein the phosphate source is P₂O₅, NaH₂PO₄, Na₂HPO₄, Na₃PO₄, KH₂PO₄, K₂HPO₄, K₃PO₄, or any combination thereof.

4. The glass composition according to claim 1, wherein the glass composition comprises:
a) from about 10 mol % to about 80 mol % of CaO;
b) from about 10 mol % to about 80 mol % of MgO;
c) from about 10 mol % to about 80 mol % of a combination of CaO and MgO;
d) from about 10 mol % to about 80 mol % of a combination of (i) CaO, and (ii) Na₂O or K₂O or both;
e) from about 10 mol % to about 80 mol % of a combination of (i) MgO, and (ii) Na₂O or K₂O or both;
f) from about 10 mol % to about 80 mol % of a combination of (i) CaO, (ii) MgO, and (iii) Na₂O or K₂O or both;
g) from about 10 mol % to about 80 mol % of a combination of (i) CaO or MgO, and (ii) a phosphate source;
h) from about 10 mol % to about 80 mol % of a combination of (i) CaO, (ii) MgO, and (iii) a phosphate source;
i) from about 10 mol % to about 80 mol % of a combination of (i) CaO or MgO, (ii) a phosphate source, and (iii) Na₂O or K₂O or both; or
j) from about 10 mol % to about 80 mol % of a combination of (i) CaO, (ii) MgO, (iii) a phosphate source, and (iv) Na₂O or K₂O or both.

5. The glass composition according to claim 1, wherein the glass composition comprises Na₂O, CaO, and MgO in a molar ratio of 1.0 to 0.5-2.5 to 0.5-2.5 (Na₂O:CaO:MgO).

6. The glass composition according to claim 5, wherein the glass composition comprises:
a) from about 16 mol % to about 22 mol % Na₂O, from about 11 mol % to about 17 mol % CaO, and from about 16 mol % to about 22 mol % MgO;
b) from about 14 mol % to about 20 mol % Na₂O, from about 14 mol % to about 20 mol % CaO, and from about 16 mol % to about 22 mol % MgO;
c) from about 11 mol % to about 17 mol % Na₂O, from about 16 mol % to about 22 mol % CaO, and from about 16 mol % to about 22 mol % MgO; or
d) from about 13 mol % to about 19 mol % Na₂O, from about 18 mol % to about 24 mol % CaO, and from about 18 mol % to about 24 mol % MgO.

7. The glass composition according to claim 1, wherein the molar ratio of (B₂O₃+MgO):(CaO+Na₂O+K₂O) is greater than 1.0, such as greater than 1.15 or greater than 1.30.

8. The glass composition according to claim 1, wherein the glass composition comprises at least 54 mol %, such as at least 57 mol %, of a combination of B₂O₃ and MgO.

9. The glass composition according to claim 1, wherein the glass composition comprises at least 33 mol %, such as at least 40 mol % or at least 50 mol %, of a combination of CaO and MgO.

10. The glass composition according to claim 1, wherein the glass composition comprises at least 7 mol %, such as at least 15 mol % or at least 30 mol %, of a combination of N₂O and K₂O.

11. The glass composition according to claim 7, wherein the glass composition comprises less than 0.1 mol % phosphate.

12. The glass composition according to claim 7, wherein the glass composition consists essentially of B₂O₃, one or both of Na₂O and K₂O, and one or both of CaO and MgO.

13. The glass composition according to claim 1, wherein the glass composition comprises B₂O₃, one or both of Na₂O and K₂O, and one or both of CaO and MgO in amounts according to any one of the compositions listed in the following table:

| B₂O₃ | Na₂O | K₂O | CaO | MgO |
|---|---|---|---|---|
| (mol %) | (mol %) | (mol %) | (mol %) | (mol %) |
| about 42 | about 5 | about 0 | about 25 | about 29 |
| about 45 | about 5 | about 10 | about 5 | about 35 |
| about 45 | about 21 | about 0 | about 22 | about 12 |
| about 40 | about 19 | about 0 | about 16 | about 25 |
| about 45 | about 25 | about 4 | about 5 | about 21 |
| about 42 | about 5 | about 5 | about 25 | about 23 |
| about 31 | about 8 | about 18 | about 20 | about 24 |
| about 45 | about 5 | about 18 | about 7 | about 25 |
| about 45 | about 15 | about 10 | about 16 | about 14 |
| about 45 | about 10 | about 6 | about 14 | about 25 |
| about 35 | about 15 | about 13 | about 12 | about 25 |
| about 43 | about 16 | about 0 | about 25 | about 16 |
| about 29 | about 25 | about 14 | about 7 | about 25 |
| about 39 | about 17 | about 12 | about 17 | about 15 |
| about 45 | about 18 | about 15 | about 10 | about 12 |
| about 45 | about 18 | about 3 | about 22 | about 12 |
| about 29 | about 18 | about 15 | about 16 | about 22 |
| about 45 | about 18 | about 0 | about 15 | about 22 |
| about 45 | about 13 | about 7 | about 13 | about 22 |
| about 45 | about 11 | about 0 | about 22 | about 22 |
| about 41 | about 14 | about 11 | about 17 | about 16 |
| about 36 | about 14 | about 15 | about 13 | about 22 |
| about 45 | about 18 | about 10 | about 5 | about 22 |
| about 35 | about 18 | about 3 | about 22 | about 22 |
| about 36 | about 5 | about 15 | about 22 | about 22 |
| about 45 | about 6 | about 15 | about 12 | about 22 |
| about 45 | about 5 | about 6 | about 22 | about 22 |
| about 42 | about 13 | about 15 | about 21 | about 9 |
| about 45 | about 5 | about 15 | about 22 | about 13 |
| about 40 | about 18 | about 15 | about 5 | about 22 |
| about 35 | about 18 | about 3 | about 22 | about 22. |

14. The glass composition according to claim 1, wherein the glass composition comprises less than 30 mol % of CaF₂ or SnF₂; and less than 30 mol % of a combination of CaF₂ and SnF₂.

15. The glass composition according to claim 1, wherein the glass composition comprises from about 2 mol % to about 15 mol % of CaF₂, SnF₂, NaF, KF, Na₂PO₃F, or a combination thereof, such as from about 5 mol % to about 15 mol % of CaF₂, NaF, KF or a combination thereof.

16. The glass composition according to claim 1, comprising: about 43 mol % of B₂O₃, about 21 mol % MgO, about 21 mol % CaO, and about 15 mol % Na₂O; such as comprising 43.0 mol % of B₂O₃, 20.7 mol % MgO, 20.7 mol % CaO, and 15.6 mol % Na₂O.

17. The glass composition according to claim 1, comprising:
from about 25 mol % to about 45 mol %, such as from about 41 mol % to about 45 mol %, of B₂O₃;
from about 10 mol % to about 23 mol %, such as from about 13 mol % to about 23 mol %, of CaO;
from about 10 mol % to about 30 mol %, such as from about 18 mol % to about 23 mol %, of MgO; and
from about 8 mol % to about 22 mol %, from about 13 mol % to about 22 mol %, of Na₂O;
and optionally from about 8 mol % to about 15 mol % of NaF, KF, CaF₂, or any combination thereof.

18. The glass composition according to claim 1, comprising:
from about 25 mol % to about 43 mol % B₂O₃;
from about 14 mol % to about 21 mol % CaO;
from about 19 mol % to about 29 mol % MgO;
from about 9 mol % to about 15 mol % Na₂O; and
from about 9 mol % to about 15 mol % of NaF, KF, CaF₂, or any combination thereof.

19. The glass composition according to claim 1, comprising:
from about 29 mol % to about 45 mol % of B₂O₃;
from about 5 mol % to about 22 mol % of CaO;
from about 1 mol % to about 22 mol % of MgO;
from 0 mol % to about 15 mol % of K₂O; and
from about 5 mol % to about 18 mol % of Na₂O.

20. The glass composition according to claim 1, wherein the glass composition comprises less than 0.1 mol % of ZnO, such as substantially no ZnO; less than 0.1 mol % of CuO; less than 0.1 mol % of Li₂O; less than 0.1 mol % of Rb₂O; less than 0.1 mol % of BaO; less than 0.1 mol % of SrO; less than 0.1 mol % of SiO₂; or any combination thereof.

21. The glass composition according to claim 1, wherein the glass composition is a particulate material that comprises particles that are from about 1 to about 50 μm in size.

22. The glass composition according to claim 21, wherein at least 75% of the particles are smaller than 50 μm in size.

23. The glass composition according to claim 21, wherein at least 85% of the particles are smaller than 50 μm in size.

24. The glass composition according to claim 21, wherein at least 95% of the particles are smaller than 50 μm in size.

25. The glass composition according to claim 21, wherein at least 5% of the particles are smaller than 7 μm in size.

26. The glass composition according to claim 21, wherein:
at least 5% of the particles are smaller than 35 μm in size,
at least 5% of the particles are smaller than 15 μm in size, and
at least 5% of the particles are smaller than 7 μm in size.

27. The glass composition according to claim 21, wherein:
at least 5% of the particles are from about 15 μm to about 35 μm in size,
at least 5% of the particles are from about 6 μm to about 15 μm in size, and
at least 5% of the particles are from about 3 μm to about 7 μm in size.

28. The glass composition according to claim 21, wherein:
about 10% of the particles are smaller than 5 μm in size,
about 50% of the particles are smaller than 15 μm in size, and
about 90% of the particles are smaller than 30 μm in size.

29. The glass composition according to claim 21, wherein the glass composition loses at least 5 mass % within 24 hours when exposed to a buffered saline solution.

30. The glass composition according to claim 21, wherein the glass composition loses at least 20 mass % within 24 hours when exposed to a buffered saline solution.

31. The glass composition according to claim 21, wherein the glass composition loses at least 40 mass % within 24 hours when exposed to a buffered saline solution.

32. The glass composition according to claim 21, wherein the glass composition loses at least 60 mass % within 24 hours when exposed to a buffered saline solution.

33. The glass composition according to claim 21, wherein the glass composition loses at least 80 mass % within 24 hours when exposed to a buffered saline solution.

34. A toothpaste comprising the glass composition according to claim 21.

35. A toothpaste comprising the glass composition according to claim 21, wherein the glass composition comprises from about 2 mol % to about 15 mol % of CaF₂, SnF₂, NaF, KF, Na₂PO₃F, or a combination thereof, such as from about 5 mol % to about 15 mol % of CaF₂, NaF, KF or a combination thereof, and wherein the toothpaste includes a sufficient amount of the glass composition to result in about 500 ppm to about 1,500 ppm of the fluoride, such as from about 750 ppm to about 1,500 ppm of the fluoride, or from about 1,000 ppm to about 1,500 ppm of the fluoride.

36. A toothpaste comprising the glass composition according to claim 21, wherein the glass composition lacks fluoride, and the toothpaste includes a sufficient amount of a fluoride source, such as sodium fluoride, to result in about 1000 ppm to about 1500 ppm fluoride.

37. A toothpaste comprising: the glass composition according to claim 21; glycerin; sodium lauryl sulphate; silica; Carbopol 940; and a flavoring agent.

38. The toothpaste according to claim 34, wherein the glass composition is about 2.5 wt % to about 7.5 wt % of the toothpaste.

39. The toothpaste according to claim 34, wherein the toothpaste is substantially water-free.

40. A prophylaxis paste comprising the glass composition according to claim 21.

41. A prophylaxis paste comprising the glass composition according to claim 21, wherein the glass composition comprises from about 2 mol % to about 15 mol % of CaF₂, SnF₂, NaF, KF, Na₂PO₃F, or a combination thereof, such as from about 5 mol % to about 15 mol % of CaF₂, NaF, KF or a combination thereof, and wherein the toothpaste includes a sufficient amount of the glass composition to result in about 1,000 ppm to about 1,500 ppm of the fluoride.

42. A prophylaxis paste comprising the glass composition according to claim 21, wherein the glass composition lacks fluoride and the prophylaxis paste includes a sufficient amount of a fluoride source, such as sodium fluoride, to result in about 1000 ppm to about 1500 ppm fluoride.

43. The prophylaxis paste according to claim 40, wherein the prophylaxis paste is substantially water-free.

44. A tooth varnish comprising the glass composition according to claim 21.

45. A tooth varnish comprising the glass composition according to claim 21, wherein the glass composition comprises from about 2 mol % to about 15 mol % of CaF₂, SnF₂, NaF, KF, Na₂PO₃F, or a combination thereof, such as from about 5 mol % to about 15 mol % of CaF₂, NaF, KF or a combination thereof, and wherein the tooth varnish includes a sufficient amount of the glass composition to result in about 1,000 ppm to about 5000 ppm of the fluoride.

46. A tooth varnish comprising the glass composition according to claim 21, wherein the glass composition lacks fluoride and the tooth varnish includes a sufficient amount of a fluoride source, such as sodium fluoride, to result in about 1000 ppm to about 5000 ppm fluoride.

47. The tooth varnish according to claim 44, wherein the tooth varnish is substantially water-free.

48. (canceled)

49. (canceled)

50. (canceled)

51. A method of at least temporarily reducing, in an individual, pain associated with sensitive teeth, the method comprising applying:
the toothpaste according to claim 34,
to dentin in the individual.

52. A glass composition according to claim 21 for desensitizing dentin.

53. (canceled)

54. A dentin-desensitizing composition comprising:
(i) the glass composition according to claim 21; and
(ii) a water-free, orally-compatible carrier.

55. The dentin-desensitizing composition according to claim 54 wherein the orally-compatible carrier is a mouthwash.

56. The dentin-desensitizing composition according to claim 54 wherein the orally-compatible carrier is formulated to mix with a mouthwash.

57. The dentin-desensitizing composition according to claim 54 wherein the orally-compatible carrier is an orally-compatible viscous carrier.

58. The dentin-desensitizing composition according to claim 57 wherein the orally-compatible viscous carrier has a viscosity from about 100 cP at 30° C. to about 150,000 cp at 30° C.

59. The dentin-desensitizing composition according to claim 58 wherein the orally-compatible viscous carrier is a toothpaste, a dental gel, a prophylaxis paste, a tooth varnish, or a bonding agent.

60. The glass composition according to claim 1, wherein the glass is a bulk glass, for preparing a particulate glass composition.

61. (canceled)

62. A method to increase surface enamel microhardness, the method comprising applying the toothpaste according to claim 34 to enamel in the individual.

63. (canceled)

64. (canceled)

65. A method to at least partially remineralize surface enamel, the method comprising applying the toothpaste according to claim 34 to enamel in the individual.

66. (canceled)

67. (canceled)

68. A method to at least partially occlude one or more dentin tubules, the method comprising applying the toothpaste according to claim 34 to the dentin tubules in an individual.

69. (canceled)
